(19) United States
(12) Patent Application Publication
(10) Pub. No.: US 2007/0167518 A1
(43) Pub. Date: Jul. 19, 2007

(54) Anti-tussive and bronchodilator actions of Enaminone E121

(76) Inventors: Ahmed Z El-Hashim, Kuwait (KW); Ivan O. Edafiogho, Kuwait (KW); Samuel B. Kombian, Kuwait (CA); Mariam H. Yousif, Kuwait (KW)

Correspondence Address: JEFFREY FURR, 253 N. MAIN STREET, JOHNSTOWN, OH, 43031, US

(21) Appl. No.: 11/307,003

(22) Filed: Jan. 18, 2006

(51) Int. Cl. A61K 31/24

(52) U.S. Cl. 514/534

(57) ABSTRACT

The invention is the new and unique use of Enaminone E121 for its significant anti-tussive and anti-bronchoconstrictor actions. E121 has direct airway smooth muscle (ASM) relaxant effect. The combined antitussive/bronchodilatory effects of E121 make the compound a novel respiratory drug that may be effective in treatment of some types of cough and also cough associated with airway flow obstruction.

### BACKGROUND OF INVENTION

This invention relates to treatment of some types of coughs and coughs associated with airway flow obstruction and in more particular by the using the enaminone E121.

### BACKGROUND

Coughs, particularly chronic coughs are a very common clinical problem with a high prevalence rate. Unfortunately, the current over-the-counter drugs provide very little therapeutic benefit, have serious side effects and yet still remain the most widely used drugs.

There is evidence to show that some types of coughs such as the non-productive chronic cough is associated with smoking, seem to improve following administration of β₂ agonists suggesting that this type of cough is bronchodilator sensitive. Additionally, patients with a non-productive cough associated with airflow obstruction seem to derive improvement in their cough following β₂ agonist treatment.

Treatment with a combination of the anti-tussive agent dextromethorphan and salbutamol has been reported to be more effective in reducing citric acid-induced cough than dextromethorphan alone. Also dextromethorphan and salbutamol were more effective in treating acute transient cough than dextromethorphan alone.

Despite this evidence, there are currently no drugs in the market with both anti-tussive and bronchodilatory action and cough is currently not treated with a combination of an anti-tussive and bronchodilator therapy perhaps due to the cost of therapy, involvement of multiple drugs, attitudes towards cough, etc.

There is still room for improvement in the art.

### SUMMARY OF THE INVENTION

The present invention relates treatment of some types of coughs and coughs associated with airway flow obstruction and in more particular by the using the enaminone E121.

The invention is the new and unique use of Enaminone E121 for its significant anti-tussive and anti-bronchoconstrictor actions. E121 has direct airway smooth muscle (ASM) relaxant effects. In addition, the ASM relaxant effects of E121 showed less tachyphalaxis than salbutomol, the standard bronchodilator. The combined antitussive/bronchodilatory effects of E121 make the compound a novel respiratory drug that is effective in treatment of some types of cough and also cough associated with airway flow obstruction.

### BRIEF DESCRIPTION OF DRAWINGS

Without restricting the full scope of this invention, the preferred form of this invention is illustrated in the following drawings:

FIG. 1 shows the Synthesis of Enaminone E121;

FIG. 2 displays E121 results;

FIG. 3 displays the summary that E121 has an anti-tussive as well as bronchoprotective effect; and

FIG. 4 displays the Summary that E121 has direct relaxant effects on ASM.

### DETAILED DESCRIPTION

The following description is demonstrative in nature and is not intended to limit the scope of the invention or its application of uses.

There are a number of significant design features and improvements incorporated within the invention.

The current invention deals with the discovery of a novel pharmacological action of the drug enaminone E121 as an anti-tussive as well as a bronchodilator. This is unique as none of the currently clinically used anti-tussive drugs have been shown to have any significant bronchodilator effects.

Enaminone E121 has significant anti-tussive and anti-bronchoconstrictor actions as shown in a guinea-pig model of citric acid induced cough and bronchoconstriction. E121 has direct airway smooth muscle (ASM) relaxant effects. In addition, the ASM relaxant effects of E121 showed less tachyphalaxis than salbutomol, the standard bronchodilator. The combined antitussive/bronchodilatory effects of E121 make the compound a novel respiratory drug that is effective in treatment of some types of cough and also cough associated with airway flow obstruction.

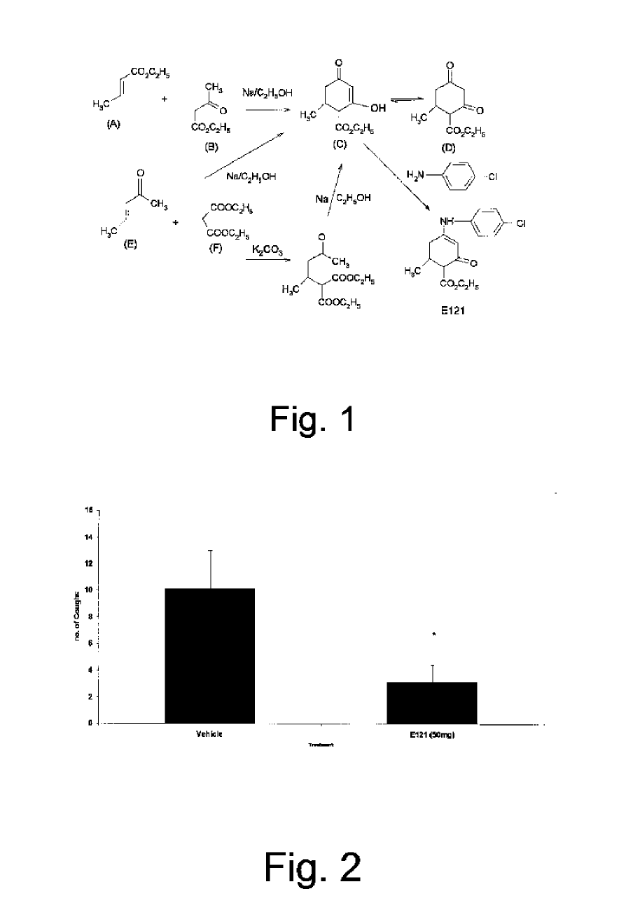

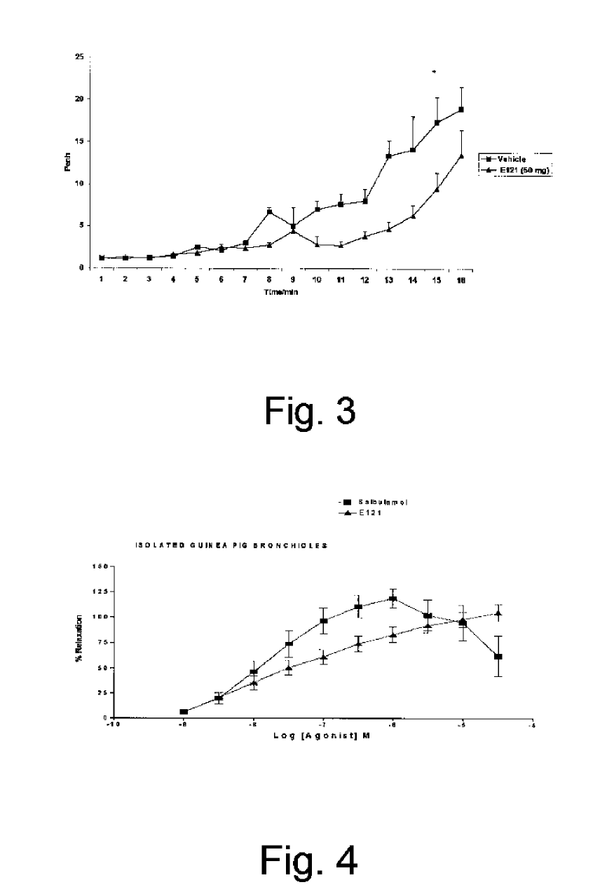

As shown in FIG. 1, the cyclization reaction between ethyl crotonate (A) and ethyl acetoacetate (B) in the presence of freshly prepared sodium ethoxide gave the intermediate beta-hydroxy keto ester (C) which existed as two tautomers (C) and (D). Condensation of (C) with 4-chloroaniline yielded the enaminone ester E121. Alternatively, pent-2-en-4-one (E) was reacted with diethyl malonate (F) in freshly prepared sodium ethoxide to give the intermediate beta-hydroxy keto ester (C). The third route involved the reaction of (E) and (F) under mild condition with potassium carbonate to give the adduct (G) which was cyclized in freshly prepared sodium ethoxide to give the intermediate beta-hydroxy keto ester (C). Thus, the synthesis of the intermediate beta-hydroxy keto ester (C) was unequivocal, and condensation with 4-chloroaniline yielded E121. The chemical nomenclature of E121 is ethyl 4-(4-chlorophenyl) amino-(-methyl-2-oxocylohex-3-en-1-oate.

The compound E121 is a cyclohexanone derivative which is a stable solid at room temperature, and has a melting point of 161-163° C. when recrystallized from ethyl acetate. It shows characteristic ultra violet and infra red absorptions, and has a molecular formula of C₁₆H₁₈NO₃Cl with a molecular weight of 307.5. It has a calculated log P(C log P) value of 3.35 using Mac log P program, version 4.0 of BioByte Corp, Claremont, Calif., USA.

The proton nuclear magnetic resonance peaks of E121 in deoterated chloroform (CDCl₃) gave chemical shifts (8 ppm) of 1.06 (d, J=6.25 Hz, 3H) for methyl group, 1.26 (t, J=6.90 Hz, 3H) for methyl group of ethyl ester, 1.90-2.70 (m, 3H) for cycloalkene ring, 3.10 (d, J=11.03 Hz, 1H) for cycloalkene ring, 4.19 (q, J=6.90 Hz, 2H) for methylene group of ethyl ester, 5.43 (s, 1H) for vinyl proton, 7.51 (s, 1H) for NH, and 7.01-7.32 (m, 4H) for phenyl ring.

Guinea pigs were placed in a transparent whole body plythesmography box with bias air flow and exposed to a nebulized aqueous solution of citric acid (0.6M). Cough was assessed visually, acoustically and by analysis of the flow signal. Bronchoconstriction was measured using enhanced pause (Penh) as an index. For more details on the methodology please refer to Effect of a novel NK1 receptor selective antagonist (NKP 60829) on citric acid induced cough and airway obstruction, Pulm Pharmacol Ther. 17(1): 11-8 by El-Hashim et al., 2004 incorporated by reference.

Protocol

Establish 2 groups

Group 1 (n=10) vehicle, i.p., 1 hr prior to citric acid challenge.

Group 2 (n=10) E121*50 mg/kg, i/p/, 1 hr prior to citric acid challenge.

Results

The results showed that E121 significantly inhibited the citric acid induced-cough (FIG. 1) and bronchoconstriction (FIG. 2).

Bronchodilatory effects of E121

Agonist-induced relaxation was studied on guinea-pig bronchial preparations pre-contracted with carbachol (3 μM). After the carbachol-induced contractions were stabilized, ascending concentrations of the agonists, salbutomol (10⁻⁹−3 ×10⁻⁴ M), or E121 (10⁻⁹−10⁻⁴ M) were added cumulatively to the organ baths to establish cumulative concentration-response curves. Inhibitory responses of the agonists were expressed as a percentage reduction of carbachol-induced tone. For full description of the methodology used in this part of the experiment, please refer to A pharmacological study of the bronchodilator properties of NKH477, forskolin and B agonists on guinea-pig and ovine isolated bronchioles, Drug. Dev. Res. 51 16-176 Yousif and Thulesius, 1999 incorporated by reference.

In the preferred embodiment, the current invention drug Enaminone E121 is ingested, however other methods of dosing can be used such as inhalators, patches or any other dosing means.

The current invention drug has many advantages over prior Art. The drug has dual action including both bronchodilation and antitussive activity. The drug exhibits significantly less tachyphalaxis than the standard bronchodilator salbutamol.

The drug did not cause any sedation at the dose used. The drug is neurotoxic.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

What is claimed is:

1. A drug comprising: Enaminone E121 as a component.

2. A drug according to claim 1, wherein said drug is used for the treatment of coughs.

3. A drug according to claim 1, wherein said drug is used for the treatment of coughs associated with airway flow obstruction.

4. A drug according to claim 1, wherein said drug is used for the treatment of respiratory problems.

5. A drug according to claim 1, wherein Enaminone E121 has a chemical nomenclature of ethyl 4-(4-chlorophenyl) amino-(-m ethyl 4-(4-chlorophenyl) amino-(-methyl-2-oxocylohex-3-en-1-oate ethyl-2-oxocylohex-3-en-1-oate.

6. A drug according to claim 1, wherein Enaminone E121 has a molecular formula of C₁₆H₁₈NO₃Cl.

7. A drug according to claim 6, wherein Enaminone E121 has a molecular weight of 307.5.

8. A drug according to claim 6, wherein Enaminone E121 has a calculated log P (C log P) value of 3.35.

9. A drug according to claim 1, wherein Enaminone E121 is ingested.

10. A drug according to claim 1, wherein Enaminone E121 is inhaled.

11. A treatment comprising: using a drug with Enaminone E121.

12. A treatment according to claim 11, wherein said treatment is for coughs.

13. A treatment according to claim 11, wherein said treatment is for coughs associated with airway flow obstruction.

14. A treatment according to claim 11, wherein said treatment is for respiratory problems.

15. A treatment according to claim 11, wherein Enaminone E121 has a chemical nomenclature of ethyl 4-(4-chlorophenyl) amino-(-m ethyl 4-(4-chlorophenyl) amino-(-methyl-2-oxocylohex-3-en-1-oate ethyl-2-oxocylohex-3-en-1-oate.

16. A treatment according to claim 11, wherein Enaminone E121 has a molecular formula of C₁₆H₁₈NO₃Cl.

17. A treatment according to claim 16, wherein Enaminone E121 has a molecular weight of 307.5.

18. A treatment according to claim 16, wherein Enaminone E121 has a calculated log P (C log P) value of 3.35.

19. A treatment according to claim 11, wherein Enaminone E121 is ingested.

20. A treatment according to claim 11, wherein Enaminone E121 is inhaled.
